(19) United States
(12) Patent Application Publication
(10) Pub. No.: US 2013/0051527 A1
(43) Pub. Date: Feb. 28, 2013

(54) IMAGE PROCESSING APPARATUS AND METHOD, AND X-RAY DIAGNOSTIC APPARATUS

(71) Applicants: Takuya SAKAGUCHI, Utsunomiya-shi (JP); Kyojiro NAMBU, Nasushiobara-shi (JP); Kunio SHIRAISHI, Otawara-shi (JP); Tooru TAKAHASHI, Nasushiobara-shi (JP)

(72) Inventors: Takuya SAKAGUCHI, Utsunomiya-shi (JP); Kyojiro NAMBU, Nasushiobara-shi (JP); Kunio SHIRAISHI, Otawara-shi (JP); Tooru TAKAHASHI, Nasushiobara-shi (JP)

(21) Appl. No.: 13/659,968

(22) Filed: Oct. 25, 2012

(30) Foreign Application Priority Data
Jul. 7, 2011 (JP) 2011-150942

(51) Int. Cl. G06K 9/00; A61B 6/00

(52) U.S. Cl. 378/62; 382/132

(57) ABSTRACT

According to one embodiment, an image processing apparatus includes a storage, a calculation unit, a generation unit and a display. The storage stores data relating to an X-ray image. The calculation unit calculates a first noise quantity of the X-ray image. The generation unit obtains a first X-ray dose corresponding to a first noise image represented by the first noise quantity based on relationship of an X-ray dose and a noise quantity, and calculates at least one second noise image corresponding to a second X-ray dose different from the first X-ray dose, and generates at least one simulated image by adding the second noise image to the X-ray image. The display displays the X-ray image and the simulated image.

### CROSS REFERENCE TO RELATED APPLICATIONS

This application is a Continuation Application of PCT Application No. PCT/JP2012/067222, filed Jul. 5, 2012 and based upon and claiming the benefit of priority from Japanese Patent Application No. 2011-150942, filed Jul. 7, 2011, the entire contents of all of which are incorporated herein by reference.

### FIELD

Embodiments described herein relate generally to an image processing apparatus and method, and an X-ray diagnostic apparatus.

### BACKGROUND

Ischemic cardiac disease is a common disease in the world. Recently, as treatment methods for this disease, intravascular treatments which are low-invasive treatments have increased in frequency from the viewpoint of reduction in burden on objects. Intravascular treatments are generally practiced under X-ray fluoroscopy, and X-ray apparatuses are used as image guide tools.

### BRIEF DESCRIPTION OF THE DRAWINGS

FIG. 1 is a block diagram illustrating an X-ray diagnostic apparatus including an image processing apparatus according to the first embodiment.

FIG. 2 is a flowchart illustrating the operation of the image processing apparatus according to the first embodiment.

FIG. 3 is (a) a graph illustrating the relationship between the noise quantity and X-ray dose, and (b) a view illustrating an example of the generation of a simulated image.

FIG. 4 is a view illustrating an example of image display according to the first embodiment.

FIG. 5 is a block diagram illustrating an image processing apparatus according to a modification of the first embodiment.

FIG. 6 is a block diagram illustrating an image processing apparatus according to the second embodiment.

FIG. 7 is a view illustrating an example of image display according to the second embodiment.

FIG. 8 is a block diagram illustrating an image processing apparatus according to the third embodiment.

FIG. 9 is a view illustrating an example of image display according to the third embodiment.

FIG. 10 is a block diagram illustrating an image processing apparatus according to the fourth embodiment.

FIG. 11 is a view illustrating an example of image display according to the fourth embodiment.

FIG. 12 is a block diagram illustrating an image processing apparatus including an image selection unit.

FIG. 13 is a block diagram illustrating an image processing apparatus according to the fifth embodiment.

FIG. 14 is a conceptual view of pulse rate setting in a rate setting unit.

### DETAILED DESCRIPTION

In a treatment under image guidance using an X-ray apparatus, however, the treatment time for a difficult case sometimes reaches two to three hours. Such a case requires extended fluoroscopy, which leads to extended exposure to radiation. This raises the problem of radiation dosage received by the patient and staff.

For this reason, there is available a technique of generating simulated images from phantoms and the like and decreasing radiation dosage by sequentially setting scan conditions based on the simulated images. In an intravascular treatment, however, the imaging direction sequentially changes relative to an object, and the state of transmission of X-rays differs depending on the object and imaging region. This makes it impossible to set X-ray doses suitable for imaging regions.

In general, according to one embodiment, an image processing apparatus includes a storage, a first calculation unit, a first generation unit and a display. The storage configured to store a data item relating to an X-ray image. The first calculation unit configured to calculate a first noise quantity of the X-ray image. The first generation unit configured to obtain a first X-ray dose corresponding to a first noise image represented by the first noise quantity, based on dependence of an X-ray dose on a noise quantity of a noise image, and to calculate at least one second noise image corresponding to a second X-ray dose different from the first X-ray dose, and to generate at least one first simulated image by adding the second noise image to the X-ray image. The display configured to display the X-ray image and the at least one first simulated image.

An image processing apparatus and method and an X-ray diagnostic apparatus according to this embodiment will be described in detail below with reference to the accompanying drawing. Note that in the following embodiments, the same reference numerals denote components which operate in the same manners, and a repetitive description will be omitted as needed.

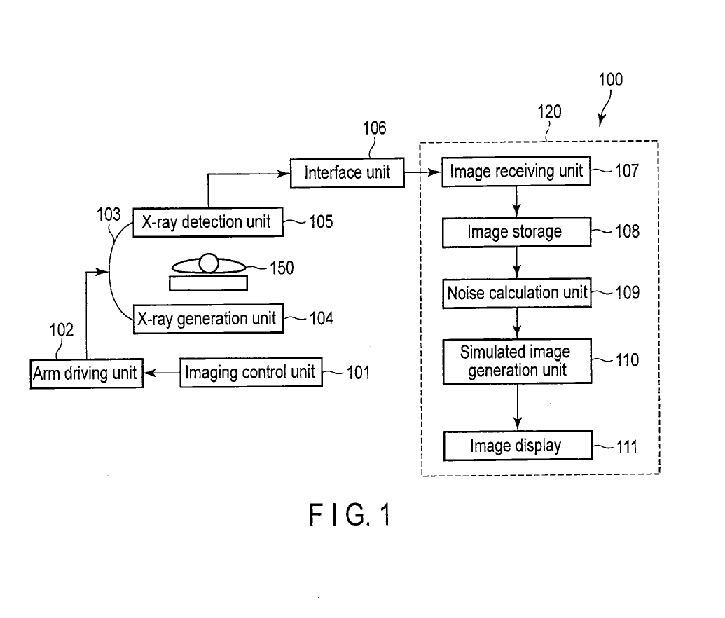

An X-ray diagnostic apparatus according to this embodiment will be described with reference to FIG. 1.

An X-ray diagnostic apparatus 100 according to this embodiment includes an imaging control unit 101, an arm driving unit 102, an arm 103, an X-ray generation unit 104, an X-ray detection unit 105, an interface unit 106, and an image processing apparatus 120.

The image processing apparatus 120 includes an image receiving unit 107, an image storage 108, a noise calculation unit 109, a simulated image generation unit 110, and an image display 111.

The imaging control unit 101 generates control signals for X-ray imaging of an object 150 in response to external triggers to control the X-ray generation unit 104, X-ray detection unit 105, and arm driving unit 102 (to be described later). Control signals are signals for deciding an X-ray dose, imaging direction, and imaging position for the execution of imaging. Assume that an X-ray dose is the quantity decided by one of imaging conditions including a tube voltage, tube current, pulse width, pulse rate, and radiation quality or a combination of them. To decide an imaging direction and an imaging position is to decide, for example, an X-ray angle of irradiation relative to the object 150, the distance between the X-ray detection unit 105 and the X-ray generation unit 104, and an X-ray imaging field of view from the X-ray generation unit 104.

The arm driving unit 102 receives a control signal from the imaging control unit 101 and rotates the arm 103 in accordance with the control signal.

The arm 103 supports the X-ray generation unit 104 at one end, and the X-ray detection unit 105 at the other end so as to make them face each other. The arm driving unit 102 can rotate the arm 103.

The X-ray generation unit 104 is supported on one end of the arm 103 and generates X-rays in accordance with a control signal from the imaging control unit 101. Note that the X-ray generation unit 104 may include an X-ray stop unit (not shown) to limit an X-ray irradiation field relative to the object 150.

The X-ray detection unit 105 is supported on the other end of the arm 103 which is different from that which supports the X-ray generation unit 104. The X-ray detection unit 105 detects the X-rays generated from the X-ray generation unit 104 and generates an X-ray image. The X-ray detection unit 105 may use, for example, a flat panel detector (FPD).

The interface unit 106 receives an X-ray image from the X-ray detection unit 105 and performs analog-to-digital conversion, protocol conversion, and the like.

The image receiving unit 107 receives an X-ray image via the interface unit 106.

The image storage 108 receives and stores data item relating to an X-ray image from the image receiving unit 107.

The noise calculation unit 109 receives an X-ray image from the image storage 108 and calculates the quantity of noise included in the X-ray image.

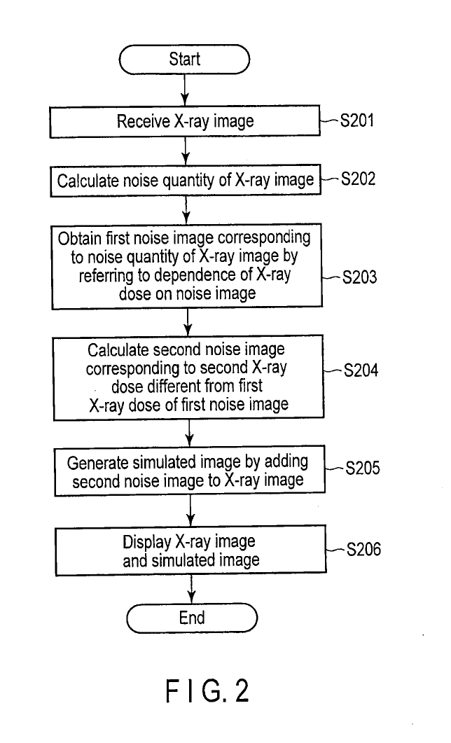

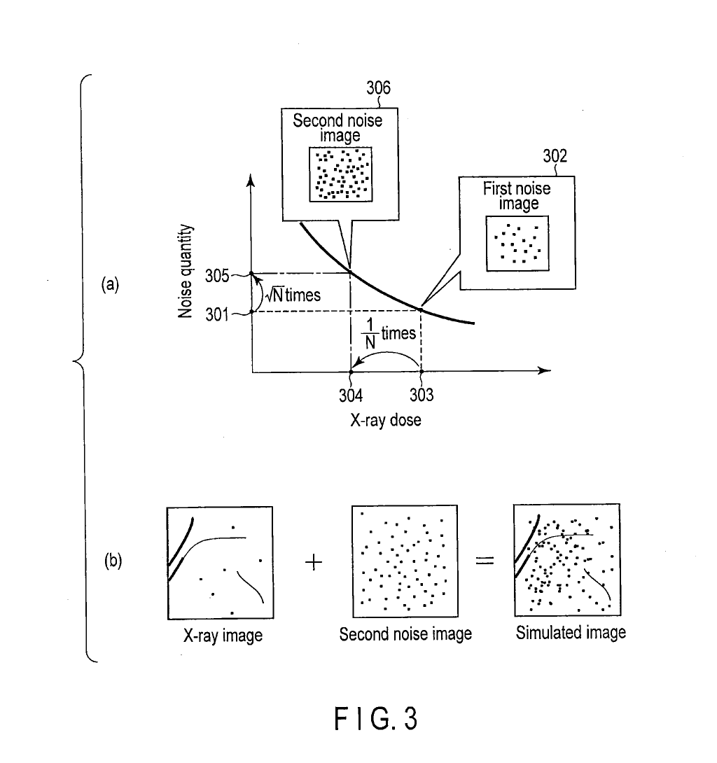

The simulated image generation unit 110 receives an X-ray image and a noise quantity from the noise calculation unit 109, and generates at least one simulated image indicating an image with an X-ray dose different from that set when an X-ray image has been captured, based on the dependence of the X-ray dose on the noise quantity of a noise image. That is, the simulated image generation unit 110 generates a simulated image with an X-ray dose smaller or larger than that set when an X-ray image has been captured. Simulated image generation processing will be described later with reference to FIGS. 2 and 3.

The image display 111 receives an X-ray image and one or more simulated images from the simulated image generation unit 110 and displays the X-ray image and one or more simulated images.

The operation of the image processing apparatus 120 will be described next with reference to the flowchart of FIG. 2.

In step S201, the image receiving unit 107 receives an X-ray image.

In step S202, the noise calculation unit 109 calculates the noise quantity (to be referred to as the first noise quantity hereinafter) of the X-ray image.

In step S203, the simulated image generation unit 110 obtains the first noise image corresponding to the noise quantity based on the dependence of the X-ray dose on the noise quantity of the noise image. The simulated image generation unit 110 also obtains the first X-ray dose corresponding to the first noise image and first noise quantity.

Dependence in this case is a calculation expression based on the noise quantity of a reference noise image. A noise image corresponding to the first noise quantity is calculated by substituting the first noise quantity in the calculation expression. It is possible to use, as a reference noise image, a noise image (average O and variance σ²) with an incident dose of 1 μR at the X-ray detection unit 105. Note that since the noise quantity and the X-ray dose vary depending on the environment or apparatus, it is preferable to generate the dependence of an X-ray dose on a noise quantity from a reference noise image for each diagnosis.

In step S204, the simulated image generation unit 110 calculates a second noise image corresponding to the second X-ray dose different from the first X-ray dose.

In a general X-ray image, since noise has a Poisson distribution, the standard deviation of noise is \sqrt{L} when L represents an image luminance. In addition, considering a dark current can perform accurate noise estimation. That is, if the X-ray dose is 1/N, the noise quantity is 1/\sqrt{N}. Therefore, the smaller the X-ray dose, the smaller the noise quantity. In practice, however, as the X-ray dose decreases, the overall luminance of the image also decreases, resulting in difficulty in viewing the image clearly. In general, therefore, the luminance of an image is kept almost constant by multiplying by a gain corresponding to a reduction in X-ray dose. If the X-ray dose decreases to ½, the noise quantity decreases to 1/\sqrt{2}. In order to keep the luminance of the image equal to that with the original X-ray dose, the gain of 2 is multiplied. The noise quantity then becomes 2×1/\sqrt{2}=\sqrt{2}. As a consequence, the noise increases relatively. Under the conditions that keep the image luminance constant, an X-ray image deteriorates in image quality, i.e., includes a relatively large noise quantity as X-ray dose decreases.

More specifically, when obtaining the second noise image with the second X-ray dose ½ the first X-ray dose, it is ideal to calculate a noise image corresponding to a noise quantity \sqrt{2} times the noise quantity of the first noise image. Note that when generating a plurality of second noise images, it is possible to generate images with different X-ray doses such as ½ and ¼ or 2 times and 4 times the first X-ray dose.

In step S205, the simulated image generation unit 110 generates a simulated image by adding the second noise image to the X-ray image. When a plurality of second noise images are generated, the simulated image generation unit 110 generates a plurality of simulated images with different X-ray doses (different noise quantities of images). For example, the simulated image generation unit 110 generates simulated images with X-ray doses ½ and ⅓ or ¼ that set when the X-ray image has been captured.

In step S206, the image display 111 simultaneously displays the X-ray image and the simulated images. Thus, the image processing apparatus 120 finishes operating. In this manner, the apparatus can generate simulated X-ray images when the X-ray dose is decreased or increased.

Note that the above steps are based on the assumption that the apparatus generates a simulated image by calculating a noise quantity for each partial region in an X-ray image, performing the above processing from step S203 to step S205, and adding the results obtained for all the partial regions. However, this embodiment is not limited to this. For example, the apparatus may equalize the overall noise of an X-ray image by performing noise equalization processing and then performing processing similar to that in step S203 and the subsequent steps. When performing noise equalization processing, the apparatus may perform inverse conversion for noise equalization processing after the generation of a simulated image.

A practical example of generating a simulated image will be described with reference to FIG. 3.

FIG. 3(a) is a graph showing the relationship between the noise quantity and X-ray dose with the luminance of an image being constant. FIG. 3(b) is a view showing an example of the generation of a simulated image.

As shown in FIG. 3(a), when a first noise quantity 301 of an X-ray image is obtained, it is possible to obtain a first noise image 302 and a first X-ray dose 303. Thereafter, it is possible to calculate a second noise image 306 from a second noise quantity 305 (\sqrt{N} times the first noise quantity 301) corresponding to a second X-ray dose 304 when the obtained first X-ray dose 303 is decreased to 1/N (N is an arbitrary integer).

Subsequently, as shown in FIG. 3(b), it is possible to generate a simulated image corresponding to a desired X-ray dose by adding the X-ray image to the second noise image.

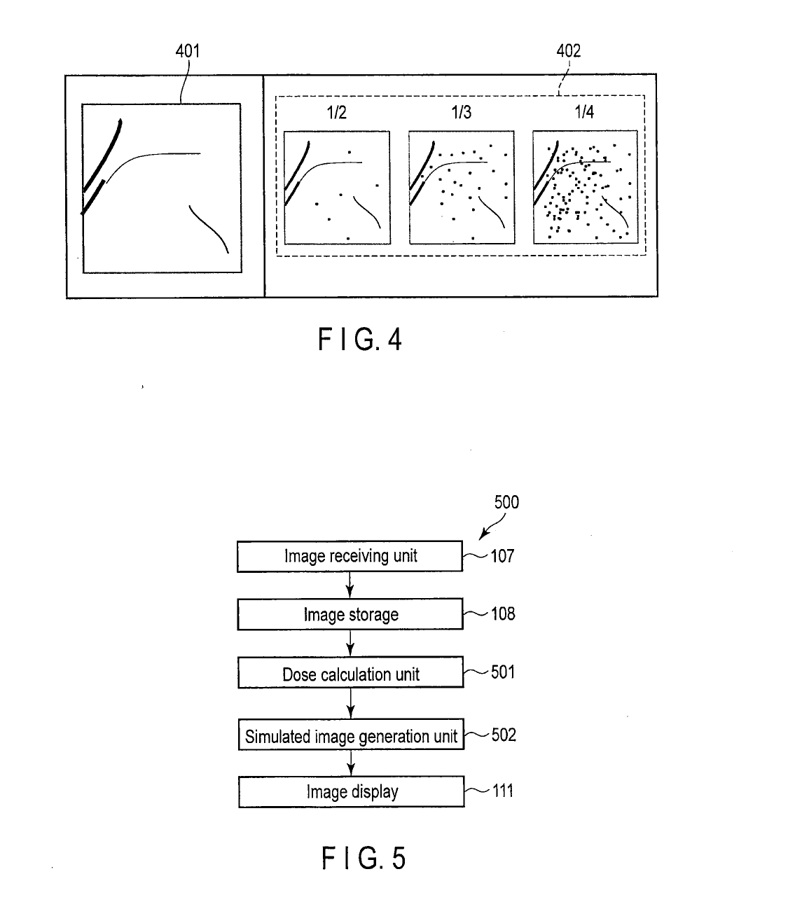

An example of image display on the image display 111 will be described next with reference to FIG. 4.

FIG. 4 shows a case in which an X-ray image 401 and three simulated images 402 with X-ray doses of ½, ⅓, and ¼ are displayed side by side. This allows the image quality of the X-ray image 401 to be instantaneously compared with the image qualities of the simulated images 402 on the assumption that the X-ray dose is decreased. The user can therefore easily determine a measure for X-ray doses in the range in which he/she can visually recognize a target, and hence can determine how much he/she can decrease the X-ray dose. This can support the operation of decreasing X-ray dosage.

More specifically, the user determines, by viewing simulated images, whether, for example, he/she can visually recognize a stent, and can easily determine that “he/she cannot recognize the stent with the image quality of a simulated image with ¼ X-ray dose, but can visually recognize it with the image quality of a simulated image with ⅓ X-ray dose”.

Note that there are three simulated images in the case shown in FIG. 4. However, this embodiment is not limited to this case, and may simultaneously display more simulated images. In addition, this case is based on the assumption that the X-ray image is displayed on the main monitor, and the simulated images are displayed on the sub-monitor. However, the X-ray image and the simulated images may be displayed in different windows within the same monitor.

In addition, simulated images may be displayed at one of the following timings: always-on display, every time an image is stored in the image storage 108, at predetermined intervals (every 30 minutes), and every time the angle of irradiation of the X-rays changes.

In this embodiment, the image receiving unit 107 may receive either still images or moving images. At this time, it is preferable to display simulated images as moving images. Note, however, that it is not necessary to process X-ray images and simulated images at the same frame rate. In addition, when displaying a moving image, it is preferable to prepare reference noise images corresponding to several frames.

In addition, this embodiment sets X-ray doses in the form of relative values (½, ⅓, and the like).

It is, however, possible to set X-ray doses in the form of absolute values (mGy and the like).

Note that when the user selects a simulated image, the apparatus may change imaging conditions so as to make an X-ray image become the selected simulated image.

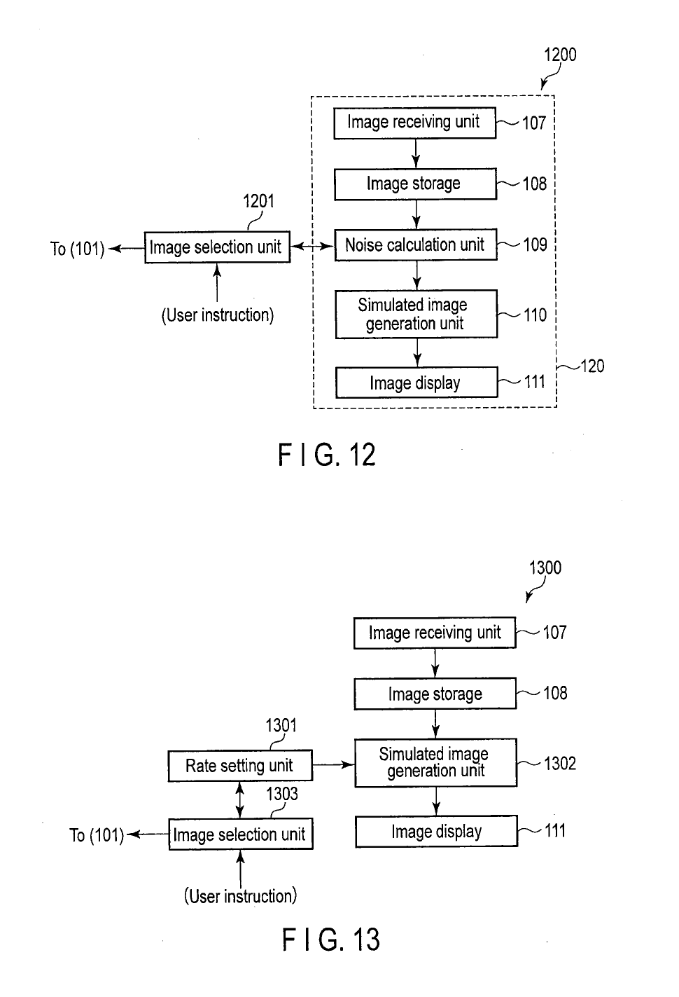

An image processing apparatus including an image selection unit will be described with reference to the block diagram of FIG. 12.

An image processing apparatus 1200 shown in FIG. 12 includes an image selection unit 1201 in addition to the image processing apparatus 120 shown in FIG. 1.

The image selection unit 1201 selects an image in accordance with an instruction from the user. As a method of selecting an image, for example, the apparatus may use a method of making the image selection unit 1201 extract coordinate information on the display screen when the user touches a simulated image displayed on the image display 111. The image selection unit 1201 may determine the simulated image displayed at the position corresponding to the coordinate information as the selected simulated image.

When the user selects a simulated image, the image selection unit 1201 receives an X-ray dose corresponding to the noise quantity of the selected simulated image from the noise calculation unit 109, and notifies the imaging control unit 101 of the imaging conditions to be set (for example, a pulse width, tube current, and rate which decide an X-ray dose).

This allows the user to set imaging conditions for obtaining a desired X-ray image by only selecting a simulated image. This makes it possible to adjust radiation dosage more easily. It is also possible to hold imaging conditions for a standard X-ray image before the selection of a simulated image and allow to return to the standard X-ray image. For example, the apparatus may display a button like “standard X-ray image” on the screen in advance and perform imaging operation under the imaging conditions for the standard X-ray image when the user touches the button.

According to the first embodiment described above, the apparatus generates simulated images by using the second noise images corresponding to the second X-ray doses different from the first X-ray dose of the current X-ray image based on the dependence of the X-ray image on the noise quantity of the X-ray image, and displays the simulated images with different X-ray doses (noise quantities) and the current X-ray image, thereby allowing the user to easily determine, by viewing the simulated images with the different noise quantities. This allows the user to easily determine how much he/she can decrease the radiation dosage while ensuring the visibility of the image. In this manner, the apparatus can support the operation of decreasing radiation dosage.

### Modification of First Embodiment

The image processing apparatus 120 described above generates simulated images based on the noise quantity of an X-ray image. However, the apparatus may calculate an X-ray dose when an X-ray image has been captured.

FIG. 5 is a block diagram of the image processing apparatus according to this modification.

An image processing apparatus 500 according to this modification includes the image receiving unit 107, the image storage 108, a dose calculation unit 501, a simulated image generation unit 502, and the image display 111.

The image receiving unit 107, the image storage 108, and the image display 111 perform the same operations as those in the first embodiment, and hence a description of them will be omitted.

The dose calculation unit 501 receives an X-ray image from the image storage 108 and calculates the first X-ray dose by calculating the luminance of the X-ray image. If the data of an X-ray image includes imaging information indicating imaging conditions (tube current, tube voltage, pulse width, and the like), the dose calculation unit 501 may calculate an X-ray dose by referring to the imaging information.

The simulated image generation unit 502 receives the first X-ray dose from the dose calculation unit 501, and calculates the second noise image corresponding to the second X-ray dose different from the first X-ray dose based on the dependence of the X-ray quantity on the noise quantity of the noise image. The simulated image generation unit 502 then generates a simulated image by adding the second noise image to the X-ray image.

According to the above modification of the first embodiment, it is possible to generate a simulated image in the same manner as in the first embodiment while decreasing the amount of processing for simulated image generation, thereby supporting the operation of decreasing radiation dosage.

### Second Embodiment

The second embodiment may present an image obtained by further performing image processing for a simulated image. This allows the user to select a more user-friendly image.

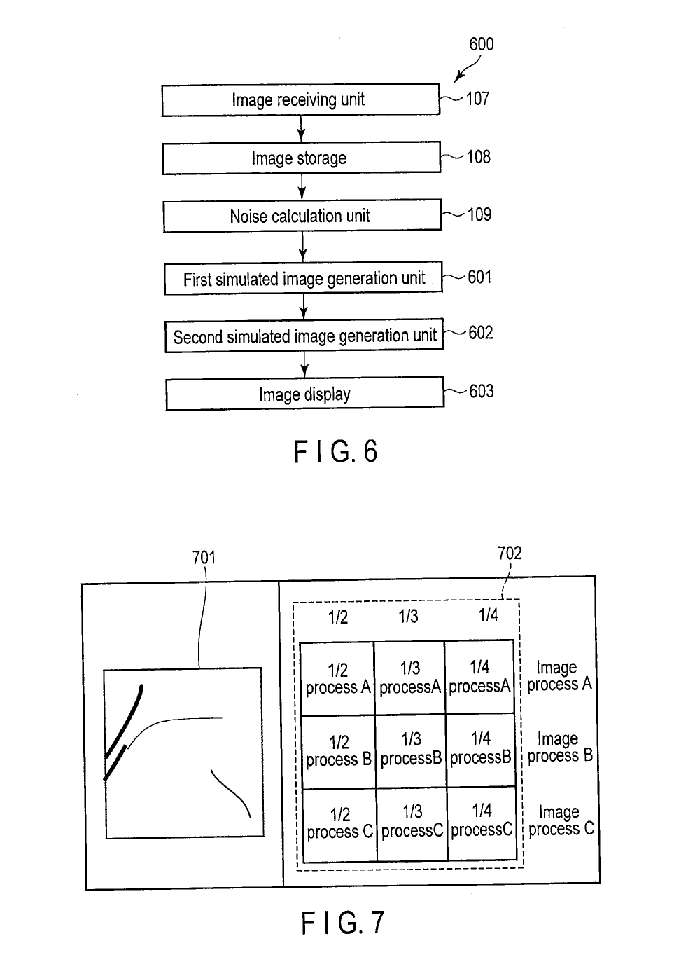

The image processing apparatus according to the second embodiment will be described with reference to the block diagram of FIG. 6.

An image processing apparatus 600 according to the second embodiment includes an image receiving unit 107, an image storage 108, a noise calculation unit 109, a first simulated image generation unit 601, a second simulated image generation unit 602, and an image display 603. Note that the image receiving unit 107, the image storage 108, and the noise calculation unit 109 perform the same processing as that in the first embodiment, and hence a description of them will be omitted.

The first simulated image generation unit 601 performs almost the same operation as that of the simulated image generation unit 110 according to the first embodiment.

The second simulated image generation unit 602 receives an X-ray image and one or more simulated images from the first simulated image generation unit 601, and obtains a plurality of simulated images by performing one or more image processes (for example, filter processes) for each of one or more simulated images. For the sake of convenience, the simulated image generated by the first simulated image generation unit 601 will be referred to as the first simulated image, and the simulated image generated by the second simulated image generation unit 602 will be referred to as the second simulated image. For example, when two filter processes are performed for three first simulated images, it is possible to obtain 3×2=6 second simulated images.

Filter processing may use a spatial filter concerning general image processing such as an edge enhancement filter or recursive filter.

The image display 603 receives an X-ray image and a plurality of second simulated images from the second simulated image generation unit 602, and displays the X-ray image and the plurality of second simulated images.

An example of image display on the image display 603 will be described next with reference to FIG. 7.

Referring to FIG. 7, the image display 603 simultaneously displays an X-ray image 701 and nine second simulated images 702 obtained by performing three filter processes including image process A, image process B, and image process C for each of the three first simulated images with X-ray doses of ½, ⅓, and ¼.

This allows the user to select an image with more suitable image quality owing to differences in image processing in addition to differences in X-ray dose. For example, viewing the display shown in FIG. 7 allows the user to “select process B with an X-ray dose of ⅓ because a dose of ½ allows image process A to be easily viewed, an X-ray dose of ⅓ allows image process B to be easily viewed, and an X-ray dose of ¼ allows image process C to be easily viewed but does not allow the stent to be viewed”.

Note that the apparatus may extract only a partial region instead of the overall second simulated image and display it on the display. In addition, for comparison, the apparatus may simultaneously display the first and second simulated images.

Furthermore, the image display 603 may include a touch panel or the like. When the user selects one of the second simulated images, the apparatus may send the selected X-ray dose to the imaging control unit 101 and send the selected image processing to the second simulated image generation unit 602.

In the second embodiment, the apparatus generates simulated images based on X-ray doses and image processing. However, the apparatus may generate simulated images with variations in imaging conditions concerning the X-ray dose. For example, the first simulated image generation unit 601 generates simulated images upon increasing and decreasing the tube current. The second simulated image generation unit 602 generates simulated images upon increasing and decreasing the pulse widths of the first simulated images. In this case, the first simulated image generation unit 601 may refer to the dependence of the noise quantity on the tube current, and the second simulated image generation unit 602 may refer to the dependence of the noise quantity on the pulse width.

According to the second embodiment described above, displaying the second simulated images obtained by performing image processing for the first simulated images allows the user to select a more user-friendly image from the simulated images, thereby supporting the operation of decreasing radiation dosage.

### Third Embodiment

The third embodiment differs from the above embodiments in that it sets a desired value in advance. A desired value is an index value concerning an X-ray dose or image and may be one of the following values: noise quantity (noise standard deviation [SD]), image signal-to-noise ratio (SNR), image level-to-noise ratio (LNR), X-ray dose, and radiation dosage.

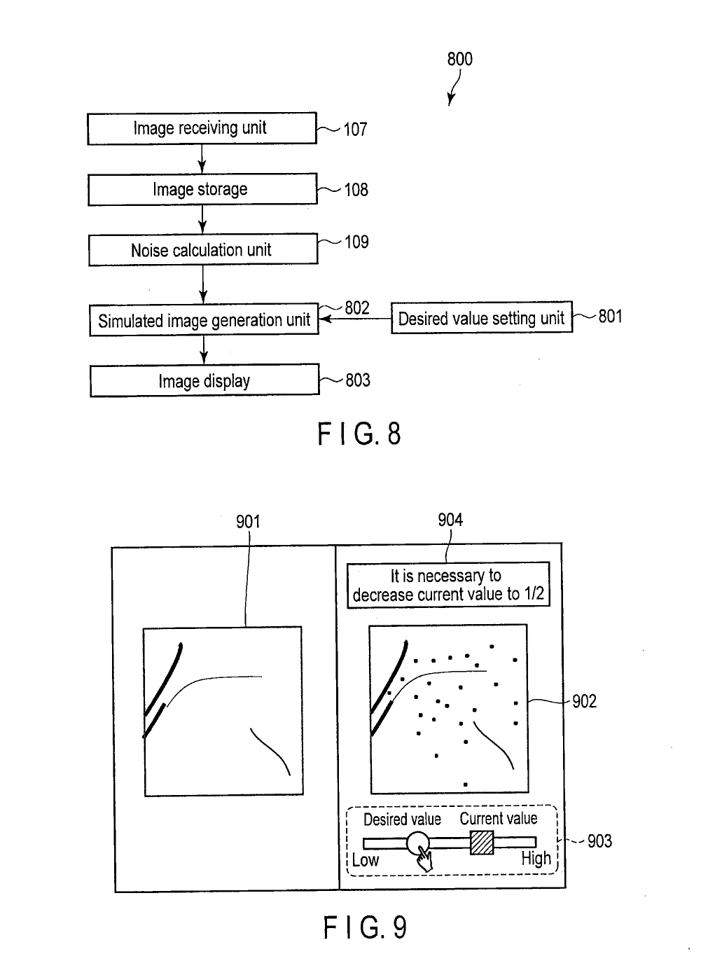

An image processing apparatus according to the third embodiment will be described with reference to the block diagram of FIG. 8.

An image processing apparatus 800 according to the third embodiment includes an image receiving unit 107, an image storage 108, a noise calculation unit 109, a desired value setting unit 801, a simulated image generation unit 802, and an image display 803. Note that the image receiving unit 107, the image storage 108, and the noise calculation unit 109 perform the same processing as that in the first embodiment, and hence a description of them will be omitted.

The desired value setting unit 801 sets a desired value relating to an image based on a user input or a specified value. It is preferable to use desired values respectively set for, for example, different facilities, environments, and objects.

The simulated image generation unit 802 performs almost the same operation as that of the simulated image generation unit 110. The simulated image generation unit 802 receives a desired value from the desired value setting unit 801, and generates a target image which is a simulated image obtained by adding a noise image with a X-ray dose corresponding to the desired value to an X-ray image. A method of generating simulated images is the same as that in the above embodiments, and a description of the method will be omitted.

The image display 803 receives an X-ray image and a target image from the simulated image generation unit 802, and simultaneously displays comments concerning the X-ray image, the target image, and the X-ray dose.

FIG. 9 shows an example of image display on the image display 803.

FIG. 9 simultaneously shows an X-ray image 901, a target image 902, a scroll bar 903, and a comment 904.

The scroll bar 903 shows the magnitude of a desired value. FIG. 9 shows a desired value as an X-ray dose, which decreases toward the left end and increases toward the right end. In addition, FIG. 9 shows the X-ray image 901 with the current X-ray dose value (the current value “□” in the scroll bar 903) and the target image 902 with an X-ray dosage value as a desired value (the desired value “∘” in the scroll bar 903). Note that the image processing apparatus 800 may change the desired value set by the desired value setting unit 801 by sliding the desired value on the scroll bar 903 to sequentially generate and display target images corresponding to desired values.

The comment 904 indicates the difference between the current value and the desired value. For example, the apparatus may display “it is necessary to further decrease the current value to ½”. In addition, the apparatus may notify the information by sound or may use both comment display and sound instead of the comment 904.

The image display 803 may display simulated images with other X-ray irradiation quantities in the above embodiment in addition to a target image. For example, the image display 803 may display simulated images concerning X-ray doses between the X-ray image and the target image.

According to the third embodiment described above, setting a desired value makes it possible to present the user, in a user-friendly manner, with an X-ray dose to be decreased. This can support the operation of decreasing radiation dosage.

### Fourth Embodiment

The fourth embodiment differs from the above embodiments in that it includes an operation unit which is used to manipulate a desired value, and performs X-ray imaging control upon deciding an X-ray dosage by using the operation unit.

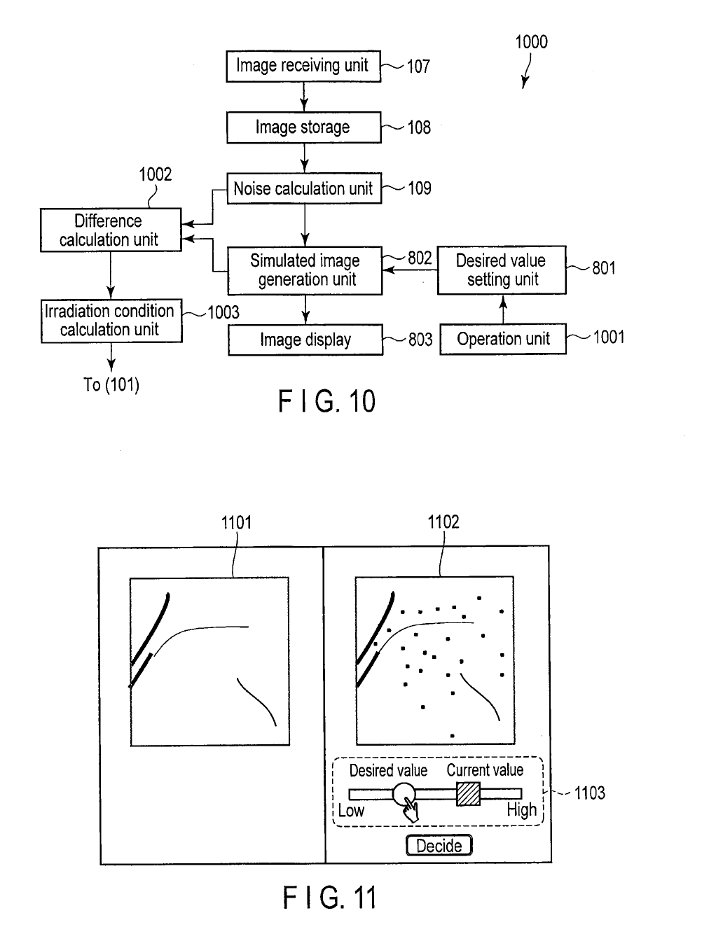

An image processing apparatus according to the fourth embodiment will be described with reference to FIG. 10.

An image processing apparatus 1000 according to the fourth embodiment includes an image receiving unit 107, an image storage 108, a noise calculation unit 109, a desired value setting unit 801, a simulated image generation unit 802, an image display 803, an operation unit 1001, a difference calculation unit 1002, and an irradiation condition calculation unit 1003.

The image receiving unit 107, the image storage 108, the noise calculation unit 109, the desired value setting unit 801, the simulated image generation unit 802, and the image display 803 perform the same operations as those in the first embodiment, and hence a description of them will be omitted.

The operation unit 1001 is, for example, a keyboard, mouse, touch panel, or the like, and accepts the input of a desired value from the user. The operation unit 1001 generates an instruction signal after input from the user or the lapse of a specified time.

The difference calculation unit 1002 receives the first noise quantity of an X-ray image from the noise calculation unit 109 and a noise quantity corresponding to a desired value from the simulated image generation unit 802, and calculates the difference between the first noise quantity and the desired value of a noise quantity.

The irradiation condition calculation unit 1003 receives the difference from the difference calculation unit 1002, and calculates X-ray irradiation conditions in X-ray imaging based on the difference. Thereafter, the irradiation condition calculation unit 1003 sends the irradiation conditions to an imaging control unit 101.

An example of image display on the image display 603 will be described next with reference to FIG. 11.

Referring to FIG. 11, the image display 603 simultaneously displays an X-ray image 1101, a target image 1102, and a scroll bar 1103. In the case shown in FIG. 11, sliding the desired value indicated on the scroll bar 1103 with the scroll bar 1103 serving as the operation unit 1001 will input a desired value to the desired value setting unit 801. Inputting the desired value from the desired value setting unit 801 to the simulated image generation unit 802 will generate a target image.

Changing the desired value in this manner can sequentially display simulated images with X-ray doses corresponding to desired values as the target images 1102. This allows the user to recognize changes in the image qualities of target images.

When the user determines to perform X-ray imaging by using a target image, the operation unit 1001 generates an instruction signal. In the case shown in FIG. 11, for example, when the user touches the target image 1102, an instruction signal is generated. Alternatively, an instruction signal is generated after the lapse of a predetermined time since a target image has been displayed on the image display 803. Note that the apparatus may use any technique such as making the user press a decision button or responding by sound as long as it is possible to decide a desired value. When an instruction signal is generated, X-ray irradiation conditions that make the X-ray image 1101 become the target image 1102 are input from the irradiation condition calculation unit 1003 to the imaging control unit 101 of the X-ray diagnostic apparatus 100 shown in FIG. 1. Under the irradiation conditions, the apparatus can perform X-ray imaging to obtain the target image.

According to the fourth embodiment described above, it is possible to easily decrease the X-ray dose and reduce the radiation dosage on an object by variably displaying target images by changing the desired value, calculating X-ray irradiation conditions corresponding to the desired value by inputting an instruction signal, and feeding back the conditions to the imaging control unit.

### Fifth Embodiment

The fifth embodiment differs from the above embodiments in that it decreases the radiation dosage on an object by changing the pulse rate when X-rays are applied.

An image processing apparatus according to the fifth embodiment will be described with reference to the block diagram of FIG. 13.

An image processing apparatus 1300 shown in FIG. 13 includes an image receiving unit 107, an image storage 108, an image display 111, a rate setting unit 1301, a simulated image generation unit 1302, and an image selection unit 1303.

The image receiving unit 107, the image storage 108, and the image display 111 perform the same operations as those in the first embodiment, and hence a description of them will be omitted.

The rate setting unit 1301 sets one or more pulse rate values different from that of a standard X-ray moving image. A pulse rate value indicates time intervals at which X-rays are applied at the time of imaging of an object.

The simulated image generation unit 1302 receives a pulse rate value from the rate setting unit 1301, and generates a simulated moving image corresponding to the pulse rate value. For example, the apparatus generates simulated moving images corresponding to a plurality of pulse rates, for example, ½ and ⅓ the general pulse rate.

The image selection unit 1303 selects a moving image in accordance with an instruction from the user, receives the pulse rate value of the selected simulated moving image from the rate setting unit 1301, and notifies the imaging control unit 101 of the pulse rate to be set.

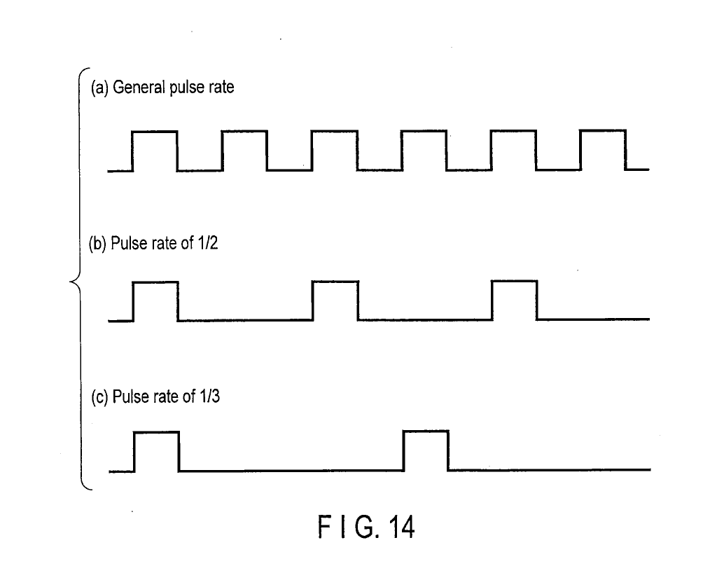

FIG. 14 is a conceptual view of the pulse rates set by the rate setting unit 1301.

FIG. 14 shows X-ray dose pulses in chronological order. Assuming that the number of frames (a) of a moving image captured at a general pulse rate is 30 frames per second, (b) represents a case in which the pulse rate is decreased to ½, that is, the frame rate is 15 frames per second, and (c) represents a case in which the pulse rate is decreased to ⅓, that is, the frame rate is 10 frames per second. As shown in FIG. 14, decreasing the frame rate of a moving image by decreasing the pulse rate can decrease the absolute value of X-ray dose on an object although the smoothness of the moving image suffers.

According to the fifth embodiment described above, when displaying an X-ray image as a moving image, the apparatus generates simulated moving images at a plurality of pulse rates. This allows the user to decrease the pulse rate within the range in which no problem occurs for a moving image in terms of smoothness. As a consequence, it is possible to decrease the radiation dosage on an object.

### Comparative Examples

A conventional technique and problems will be described as a comparative example.

As a conventional technique, there is available a technique concerning CT, which aims at decreasing radiation dosage. This conventional technique is designed to generate a “simulated image”, upon inputting of a “sample image” of a phantom or the like and desired “scan conditions”, by processing the sample image so as to comply with the corresponding noise quantity. The user can decrease the radiation dosage by sequentially re-setting the scan conditions up to the limit at which a lesion can be checked, while visually recognizing the simulated image. Using this conventional technique for intravascular treatments using an X-ray diagnostic apparatus will raise the following problems.

Problem 1: Since the user needs to sequentially re-set the “scan conditions” while visually recognizing the “simulated image”, a process of trial and error is required. That is, it takes much time to complete setting operation. Time is crucial when providing an intravascular treatment. The time required for such a process of trial and error is undesirable for this treatment.

Problem 2: In the conventional technique, a tomographic image is three-dimensional data. In contrast, images used in an intravascular treatment using an X-ray diagnostic apparatus are two-dimensional data. CT requires only one type of “sample image” to be prepared as an input. In contrast, an X-ray diagnostic apparatus requires sample images in all the directions (for example, 360 types of sample images at 1° intervals in the left anterior oblique (LAO) and right anterior oblique (RAO) directions×360 types of sample images at 1° intervals in the cranial (CRA) and caudal (CAU) intervals=129600 types), which is not practical.

Problem 3: A “sample image” used in the conventional technique is a phantom image or an image of other person, which differs from an image of the patient. That is, such an image lacks the sense of reality. In particular, the state of an object to be treated in an intravascular treatment often differs from sample conditions. If, for example, the object has cardiomegaly, image density and the like exhibit large differences.

Problem 4: If an X-ray image of the object is used instead of a “sample image” to solve the problem 3, since the value of “image SD” of the image cannot be obtained, no simulation can be done.

Problem 5: The conventional technique is not based on the premise that a moving image is input as an X-ray image, that is, new frames are sequentially input almost in real time.

Problem 6: When adjusting the image quality (and SNR and radiation dosage) of an object which the user wants to view in consideration of the visibility of the object, noise looks different between a still image and a moving image. The conventional technique is targeted at still images, and gives no consideration to the image quality of moving images.

As described above, this technique is used in environments different from an environment in which an intravascular treatment is being done and for different needs, and has the above problems from the viewpoint of high-speed, automatic, real-time operation.

This disclosure can be used for an X-ray apparatus which handles two-dimensional projection images, can handle the data of an object as inputs, and can cope with real-time input, and hence can solve the above problems.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus, comprising:
a storage configured to store a data item relating to an X-ray image;
a first calculation unit configured to calculate a first noise quantity of the X-ray image;
a first generation unit configured to obtain a first X-ray dose corresponding to a first noise image represented by the first noise quantity, based on dependence of an X-ray dose on a noise quantity of a noise image, and to calculate at least one second noise image corresponding to a second X-ray dose different from the first X-ray dose, and to generate at least one first simulated image by adding the second noise image to the X-ray image; and
a display configured to display the X-ray image and the at least one first simulated image.

2. The apparatus according to claim 1, further comprising a second generation unit configured to obtain one or more second simulated images by performing at least one image process for the at least one first simulated image,
wherein the display displays the X-ray image and the second simulated images.

3. The apparatus according to claim 1, further comprising a setting unit configured to set a desired value relating to an X-ray dose or an image,
wherein if the desired value is set, the first generation unit generates a third noise image with an X-ray dose corresponding to the desired value based on the dependence, and generates at least a target image by adding the third noise image to the X-ray image, and the display displays at least the X-ray image and the target image.

4. The apparatus according to claim 1, wherein the first generation unit generates, if a plurality of first simulated images are generated, the plurality of first simulated images with different X-ray doses each other.

5. The apparatus according to claim 4, further comprising:
a second calculation unit configured to calculate a difference between a noise quantity of the X-ray image and a noise quantity of the target image if instruction signal is input; and
a third calculation unit configured to calculate an X-ray irradiation condition from the difference to make the X-ray image become the target image.

6. The apparatus according to claim 1, further comprising a selection unit configured to select the first simulated image in accordance with an instruction from a user, and to notify an imaging condition for the selected first simulated image.

7. An image processing apparatus, comprising:
a storage configured to store a data item relating to an X-ray image;
a first calculation unit configured to calculate a first X-ray dose when the X-ray image is captured;
a first generation unit configured to calculate at least one noise image corresponding to a second X-ray dose different from the first X-ray dose, based on dependence of an X-ray dose on a noise quantity of a noise image, and to generate a first simulated image by adding the noise image corresponding to the second X-ray dose to the X-ray image; and
a display configured to display the X-ray image and the at least one first simulated image.

8. The apparatus according to claim 7, further comprising a second generation unit configured to obtain one or more second simulated images by performing at least one image process for the at least one first simulated image,
wherein the display displays the X-ray image and the second simulated images.

9. The apparatus according to claim 7, further comprising a setting unit configured to set a desired value relating to an X-ray dose or an image,
wherein if the desired value is set, the first generation unit generates a third noise image with an X-ray dose corresponding to the desired value based on the dependence, and generates at least a target image by adding the third noise image to the X-ray image, and the display displays at least the X-ray image and the target image.

10. The apparatus according to claim 7, wherein the first generation unit generates, if a plurality of first simulated images are generated, the plurality of first simulated images with different X-ray doses each other.

11. The apparatus according to claim 9, further comprising:
a second calculation unit configured to calculate a difference between a noise quantity of the X-ray image and a noise quantity of the target image if instruction signal is input; and
a third calculation unit configured to calculate an X-ray irradiation condition from the difference to make the X-ray image become the target image.

12. The apparatus according to claim 7, further comprising a selection unit configured to select the first simulated image in accordance with an instruction from a user, and to notify an imaging condition for the selected first simulated image.

13. An image processing method, comprising:
storing a data item relating to an X-ray image in a storage;
calculating a first noise quantity of the X-ray image;
obtaining a first X-ray dose corresponding to a first noise image represented by the first noise quantity, based on dependence of an X-ray dose on a noise quantity of a noise image, and to calculate at least one second noise image corresponding to a second X-ray dose different from the first X-ray dose, and generating at least one first simulated image by adding the second noise image to the X-ray image; and
displaying the X-ray image and the at least one first simulated image.

14. The method according to claim 13, further comprising obtaining one or more second simulated images by performing at least one image process for the at least one first simulated image,
wherein the displaying displays the X-ray image and the second simulated images.

15. The method according to claim 13, further comprising setting a desired value relating to an X-ray dose or an image,
wherein if the desired value is set, the generating the at least one first simulated image generates a third noise image with an X-ray dose corresponding to the desired value based on the dependence, and generates at least a target image by adding the third noise image to the X-ray image, and the displaying displays at least the X-ray image and the target image.

16. The method according to claim 13, wherein the generating the at least one first simulated image generates, if a plurality of first simulated images are generated, the plurality of first simulated images with different X-ray doses each other.

17. The method according to claim 15, further comprising:
calculating a difference between a noise quantity of the X-ray image and a noise quantity of the target image if instruction signal is input; and
calculating an X-ray irradiation condition from the difference to make the X-ray image become the target image.

18. The method according to claim 13, further comprising selecting the first simulated image in accordance with an instruction from a user to notify an imaging condition for the selected first simulated image.

19. An X-ray diagnostic apparatus, comprising:
a first generation unit configured to generate X-rays;
a detection unit configured to obtain an X-ray image by detecting X-rays applied from the first generation unit and transmitted through an object;
a control unit configured to control an X-ray angle of irradiation, position and range between the first generation unit and the detection unit relative to the object;
a storage configured to store the X-ray image;
a first calculation unit configured to calculate a first noise quantity of the X-ray image;
a setting unit configured to set a desired value relating to an X-ray dose or an image;
a second generation unit configured to calculate a noise image corresponding to a second noise quantity which relates to the desired value or corresponding to an X-ray dose which relates to the desired value, based on dependence of an X-ray dose on a noise quantity of a noise image, and to generate a target image by adding the noise image with the second noise quantity to the X-ray image;
a display configured to display the X-ray image and the target image;
a second calculation unit configured to calculate a difference between the first noise quantity and the second noise quantity if an instruction signal is input; and
a third calculation unit configured to calculate an X-ray irradiation condition from the difference to make the X-ray image become the target image,
wherein the control unit controls to perform X-ray imaging under the X-ray irradiation condition.

20. An image processing apparatus, comprising:
a storage configured to store a data item relating to an X-ray moving image;
a setting unit configured to set at least one pulse rate value indicating time intervals at which X-rays are applied when capturing the X-ray moving image;
a first generation unit configured to generate a simulated moving image upon changing a frame rate of the X-ray moving image in accordance with the pulse rate value; and
a display configured to display the X-ray moving image and the at least one simulated moving image.

21. The apparatus according to claim 20, further comprising a second generation unit configured to obtain one or more second simulated moving images by performing at least one image process for the at least one first simulated moving image,
wherein the display displays the X-ray image and the second simulated moving images.

22. The apparatus according to claim 20, further comprising a selection unit configured to select the first simulated moving image in accordance with an instruction from a user, and to notify an imaging condition for the selected first simulated moving image.
